(19) United States
(12) Patent Application Publication
(10) Pub. No.: US 2010/0222691 A1
(43) Pub. Date: Sep. 2, 2010

(54) Method and Means for Indication of Life Function

(76) Inventor: Nina Widfeldt, Invercargill (NZ)

Correspondence Address: Polster, Lieder, Woodruff & Lucchesi, L.C., 12412 Powerscourt Dr. Suite 200, St. Louis, MO, 63131-3615, US

(21) Appl. No.: 11/909,754

(22) PCT Filed: Apr. 10, 2006
(86) PCT No.: PCT/SE2006/000419

§ 371 Date: Sep. 26, 2007

(30) Foreign Application Priority Data
Apr. 18, 2005 (SE) 0500855-2

(51) Int. Cl. A61B 5/08

(52) U.S. Cl. 600/532

(57) ABSTRACT

The present invention refers to a method and a device for indication of the percentage of carbon dioxide in a person's exhaled air, when breathing occurs through a means conducting the air flow. According to the invention, a means for determining the percentage of carbon dioxide is fixed or removably connected to the means conducting the air flow. The percentage of carbon dioxide is established with regard to a lower and/or an upper boundary during determined time intervals. In presented designs of the invention, the lower boundary is 2% and the upper boundary is 7% and the time interval is 2-10 seconds. The device contains, for example, a means to indicate if the upper boundary is exceeded or if the value falls below the lower boundary at any point during the entire time interval, which can be implemented through an alarm with an audio or light signal.

The present invention refers to a device that indicates the vital function of given individuals.

The metabolism of the human body assumes that oxygen in inhaled air diffuses from the alveoli of the lungs to the bloodstream, in order to be transported to the body's cells. Cellular respiration results in the production of carbon dioxide (CO₂), which will travel in the blood to the lung circulation, and there it diffuses out to the air in the alveoli so that it can be expelled during exhalation. The occurrence of carbon dioxide in exhaled air is the only completely unambiguous sign of the occurrence of cellular metabolism, blood flow and alveolar ventilation. An adult healthy person at rest has a breathing frequency of 12-15 breaths per minute. When exhalation starts, the percentage of carbon dioxide will rise from 0% to a maximal value of carbon dioxide called the end-tidal carbon dioxide value (ETCO₂) which is normally between 4.5 and 6%. (Here and in the following, it is the per cent by volume that is considered.)

The breathing process can, in a simplified way, be divided into three phases. During the first phase, called phase 0, the inhalation of air containing an insignificant amount of carbon dioxide occurs. When exhalation starts, phase 1, the percentage of carbon dioxide in the exhaled air will rapidly rise, even if the rise occurs with a short delay, which depends on the existence of air in the bronchi that has not been in contact with the alveoli and thus does not contain any carbon dioxide. At the end of the exhalation, phase 2, the highest value of carbon dioxide, ETCO₂, is measured, after which inhalation starts again and the percentage of carbon dioxide in the breathed air returns to the 0 level.

During physical exertion, breathing frequency increases and the value of ETCO₂ can rise slightly, due to the fact that the metabolism of the working muscle cells has increased and thus the percentage of carbon dioxide in the blood can increase. Small children normally have a higher breathing frequency than adults, but the percentage of carbon dioxide during exhalation is more or less the same as for adults.

If carbon dioxide cannot be measured in exhaled air, this may be due to failing circulation to the cells of the body, inadequate blood circulation through the lung system or inadequate ventilation of the alveoli.

All living human beings generate carbon dioxide in exhaled air, which reaches a highest end-tidal value of carbon dioxide. Exhalation occurs at least ten times per minute; ETCO₂ is normally of 4.5-6%, and under normal circumstances the value will not fall below 2%.

Some examples of situations when awareness of carbon dioxide in the exhaled air may be of great importance can be mentioned. Tragic incidents exist within emergency medical care when persons die due to incorrect treatment. A situation when this can happen is when a person cannot, for some reason, maintain free bronchi without the performance of a so-called intubation. Intubation means that a tube or pipe is inserted into the trachea and that the patient is then ventilated through this tube, so that the lungs are supplied with oxygen-rich air. During the intubation, the tube can be placed by mistake in the oesophagus instead of the trachea, which means that the air does not reach the lungs. This will result in the air being forced down into the stomach, causing vomiting, with a consequential risk of suffocation. Furthermore, acid stomach content can then reach the lungs and cause chemical lung damage.

The reason for the occurrence of such events lies in the fact that the entrance to the trachea lies hidden behind the tongue and epiglottis, which makes it difficult to obtain a clear view when the tube is inserted into the throat. Furthermore, the oesophagus lies completely behind the entrance to the trachea, and it is easy to place the tube in the wrong part of the throat by mistake. Even if very sophisticated means exist to facilitate intubation, it is often hard to see the entrance to the trachea clearly. One is sometimes constrained to insert the tube “blind”. In this case, it is important to verify, without any delay, that the tube has been placed in the right part of the throat, which can be done through observation of movements in the thoracic wall at ventilation, auscultation of breathing sounds and/or a measurement device that shows the occurrence of carbon dioxide in the exhaled air.

When a person is breathing normally, the amount of carbon dioxide in the exhaled air (ETCO₂) will correspond to the amount of the carbon dioxide in the venous blood. It may be said that ETCO₂ is the only certain sign that circulation and ventilation of the lungs is occurring.

In certain physical conditions which require pain alleviation, it is possible to reach a situation where, due to the administration of a painkiller, for example morphine, depression of the respiratory centre occurs, leading to the patient breathing too slowly and not sufficiently deeply. This can result in the increase of the percentage of carbon dioxide in the blood, and thus the percentage of carbon dioxide in the exhaled air will exceed the 6% considered normal. If this respiratory depression is discovered and dealt with in time, before symptoms of inadequate oxygenation arise, this is an obvious improvement in the safety of people treated with morphine preparations during acute pain conditions, for example postoperatively or during accidents.

In the case of a serious event with a discharge of dangerous substances, medical care and rescue staff will face many difficult tasks. Discharge of dangerous substances can occur during accidents in industry, during transport accidents or in connection with war or criminal actions.

The physician and other nursing staff will then face the task of rapidly determining whether apparently lifeless persons are still alive. Many of the affected may belong to the rescue services, the police or the military and therefore may be equipped with personal protection equipment with various types of protective masks. It can be very difficult to ascertain rapidly whether a person in protective equipment is still alive or should be given priority for care, especially in a situation with many victims. If the victims are wearing some kind of breathing protection, this protection can naturally not be removed while they remain in a contaminated atmosphere. If it were possible to rapidly measure the occurrence of carbon dioxide in the exhaled air, this would be a way to decide whether a person in protective equipment is alive.

Knowledge of the percentage of carbon dioxide in a person's exhaled air can thus have an essential significance in different situations.

The purpose of the present invention is to make a device with which the percentage of carbon dioxide in the exhaled air can be rapidly and simply determined during rescue work or on other occasions where a rapid and simple assessment of vital function is essential.

A device according to the invention, as well as the designs of such a device, have the characteristic properties given in the patent requirements.

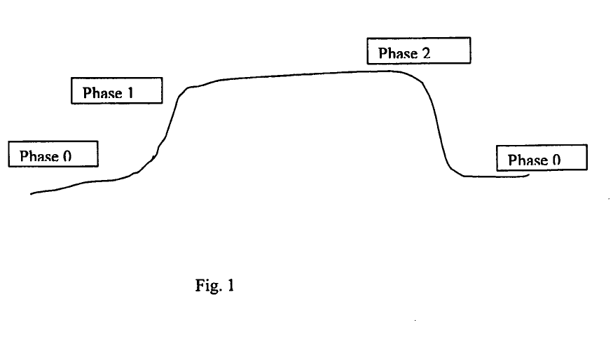

The percentage of carbon dioxide as a function of time in normal breathing is shown in FIG. 1, in which the previously mentioned breathing phases, phase 0-phase 2, are indicated with corresponding numbers.

The percentage of carbon dioxide in a person's exhaled air, when breathing occurs through a means that conducts the air flow, is measured according to the invention with regard to an upper and/or a lower boundary and during determined time intervals, and deviation from the given boundary values is indicated. Deviation can mean, for example, that a certain lowest value of the percentage of carbon dioxide is not reached during the given time interval. A device for determining the percentage of carbon dioxide in a person's exhaled air, when breathing occurs through a means that conducts the air flow, is constituted, according to the invention, by a means for determining the percentage of carbon dioxide, which is fixed or removably connected to the means conducting the air flow. The percentage of carbon dioxide is determined with regard to a lower and/or an upper boundary during determined time intervals. In a presented design, the lower boundary is 2% and the upper boundary is 7%; these boundary values are selected with respect to the previously mentioned normal values of the percentage of carbon dioxide and ETCO₂.

The time interval is preferably 2-10 seconds. A normal breathing pattern involves an exhalation occurring every 6 seconds. The device even contains a means of indicating if given boundaries are exceeded or passed within the time interval. Preferably, the indicator contains an alarm with an audio or light signal arranged in such a way that an alarm is triggered if the percentage of carbon dioxide exceeds or falls below the given boundary value during an entire period of time. If the lower boundary value is 2%, this means that the alarm is given if 2% CO₂ in the exhaled air is not reached at any point during the time period. Simultaneously with the alarm function, the indication can also occur in a different manner, for example an analogue or digital display of the instantaneous or time-integrated value of the percentage of carbon dioxide during the exhalation phase, graphic representation of the percentage of carbon dioxide or the time-integrated value on a screen, in order to show the breathing process or to show an average value of the percentage of carbon dioxide during the time interval.

The means through which the air flow passes can be an intubation tube or a breathing mask.

1. Device for indication of a person's vital function, characterized in that it has at least one device for determining the percentage of carbon dioxide in the air flow from the person's respiratory organs, as well as at least one indicator which indicates if the percentage of carbon dioxide falls below or exceeds the fixed boundary values during a determined time interval.

2. Device according to claim 1, characterized in that the lower and upper boundary values are 2% and 7% respectively, as well as the time interval being 2-10 seconds.

3. Device according to claim 1, characterized in that the device is fixed or removably connected to means for guaranteeing free bronchi.

4. Device according to claim 1, characterized in that the indicator contains an alarm with an audio or light signal, which is triggered if the percentage of carbon dioxide has not exceeded the given lower boundary value at any point during an entire time interval, or if the percentage of carbon dioxide has exceeded the given upper boundary value at any point during an entire time interval.
